(19) United States
(12) Patent Application Publication
(10) Pub. No.: US 2025/0049816 A1
(43) Pub. Date: Feb. 13, 2025

(54) COMPOSITIONS COMPRISING THYMOQUINONE AND VITAMIN D

(71) Applicant: N.S. Oils Ltd., Kibbutz Sa'ad (IL)

(72) Inventor: Mor ZEILKHA, Ramat Gan (IL)

(21) Appl. No.: 18/697,427

(22) PCT Filed: Sep. 30, 2022
(86) PCT No.: PCT/IL2022/051043

§ 371 Date: Mar. 29, 2024

Related U.S. Application Data
(60) Provisional application No. 63/251,196, filed on Oct. 1, 2021.

(51) Int. Cl. A61K 31/592; A61K 31/122; A61K 31/593; A61K 36/71; A61P 29/00

(52) U.S. Cl. CPC A61K 31/592; A61K 31/122; A61K 31/593; A61K 36/71; A61P 29/00

(57) ABSTRACT

The present invention provides a composition comprising thymoquinone (TQ) and vitamin D, wherein the weight ratio of said TQ to said vitamin D is in the range of 1:0.0001-1:1. Preferably, the composition also comprises free fatty acids, at a total concentration of 3% w/w or less. The invention is also directed to dosage forms comprising the aforementioned composition, and to various methods of treatment involving the administration of said composition and dosage forms.

### FIELD OF THE INVENTION

The present invention is directed to compositions comprising both thymoquinone and vitamin D. More specifically, the composition of the present invention comprises thymoquinone and vitamin D combined in ratios which cause synergistic enhancement of certain biological activities of one or both of those substances.

### BACKGROUND OF THE INVENTION

Vitamin D is the name given to a group of fat-soluble secosteroids responsible for many biological effects. In humans, the most important compounds in this group are vitamin D₃ (also known as cholecalciferol) and vitamin D₂ (ergocalciferol).

Although vitamin D may be found in some food substances (notably certain fatty fish and commercial cow's milk that has been supplemented with vitamin D), humans largely obtain vitamin D from exposure to sunlight via a reaction that takes place in the lower layers of the epidermis. However, the synthesis of vitamin D by means of sun exposure is insufficient in many climatic regions of the world. In addition, in many cultures, the amount of time spent outdoors exposed to the sun has been significantly reduced over the course of time. As a result, many people may have sub-optimal levels of vitamin D. Increasingly, and particularly as a result of the large number of reports concerning the protective effects of vitamin D with regard to many different disease states, many people increase their overall vitamin D levels by means of supplementation with various vitamin D-containing formulations.

While, historically, vitamin D has been associated primarily with calcium homeostasis and metabolism, it has been also been found to possess a very large number of other biological activities, including supporting immune system function, brain, and nervous system health, regulating insulin levels and insulin receptor sensitivity, supporting lung function and cardiovascular health. In addition, there is also evidence concerning the ability of vitamin D to affect the expression of specific genes involved in carcinogenesis.

Metabolic syndrome is the term used to describe a cluster of several different parameters (at least three of: abdominal obesity, elevated blood pressure, high blood glucose, high blood triglycerides and low levels of HDL-cholesterol), and which is associated with diseases such as type-2 diabetes and coronary heart disease. There are many complex biochemical and cellular mechanisms involved in the development of such diseases following the onset of metabolic syndrome. Many of these mechanisms have been found to implicate various cytokines and other signaling molecules, some of which (including, for example TNF-alpha) are also associated with the immune system and inflammation. Increasingly, inflammatory processes are being implicated in the pathogenesis of atherosclerotic disease, as well as in the development of insulin resistance and diabetes. One example of this is seen in relation to the progression from obesity to insulin resistance (i.e., a state in which target cells are refractory to the normal physiological effects of insulin following its binding to the insulin receipt on the surface of said cells). The results of some studies suggest that one general mechanism may be as follows: elevated blood sugar, leading to elevated blood triglycerides, leading to accumulation of visceral fat, leading to increased production of TNF-alpha from adipocytes, resulting in insulin resistance.

There has been a great increase in the number of cases of metabolic syndrome over the past few decades, probably as a result of unfavorable changes in diet and exercise habits. In fact, for the first time in history, there are now more people living in countries where mortality is associated with being overweight or obese, than in countries where most of the population are under-nourished and thus underweight.

It is thus a matter of the utmost importance to control the above-described parameters used to define metabolic syndrome, in individuals and in populations, in order to reduce the morbidity and excess mortality associated therewith. While changes in dietary behavior and exercise are arguably the most important immediate steps that need to be taken in obese individuals, or in those having diabetes or pre-diabetes, such strategies are often insufficient when used alone. Thus, in many cases, the patients are often also given various pharmacological agents, including anti-hyperglycemic drugs and statins and other cholesterol-lowering medications. As in the case of most powerful drug treatments, many of these agents are associated with side effects, some of which reduce patient compliance, and others which may even be life-threatening. As an alternative to drug treatment, some patients and practitioners also turn to nutraceutical (natural product) compositions, at least at the earlier stages of metabolic syndrome, in an attempt to stave off the development of more serious disease. While there are many studies showing advantageous effects associated with supplementation with various natural substances (including vitamins, carotenoids, polyphenols etc.), in some cases, such treatments are unable to normalize some of the altered lipid and/or glucose parameters that characterize metabolic syndrome.

A need thus exists for compositions of natural origin, having few or no undesirable effects, but which are able to effectively prevent or normalize the disturbances in body weight, blood glucose and lipid levels, and to support the immune system and prevent or reduce inflammatory processes.

The present invention meets this need.

### SUMMARY OF THE PRESENT INVENTION

The present invention is primarily directed to a composition comprising a combination of thymoquinone (TQ) and vitamin D. This composition may be used to prevent or treat many of the changes associated with metabolic syndrome, such as insulin resistance and increased visceral fat—by means of supporting healthy weight management—as well as preventing inflammation and providing support for the immune system.

Preferably, the molar or weight ratio between the TQ and vitamin D in the composition of the present invention is such that there is a synergistic interaction between these two components, with regard to one or more of the biological effects of one or both of them. However, in some other embodiments, such a synergistic interaction is not seen.

In one preferred embodiment, the composition of the present invention may further comprise one or more free fatty acids, wherein the weight ratio of TQ to said free fatty acids is at least 1:1.5, preferably at least 1:1, and more preferably at least 1:0.6 (wherein “at least” refers to the weight of TQ in relation to the weight of the free fatty acids, i.e., the value of the ratio is as stated or numerically larger).

In another preferred embodiment, the composition of the present invention further comprises free fatty acids (in addition to TQ and vitamin D), wherein the concentration of TQ is at least 2% w/w and the concentration of said free fatty acids is 3% w/w or less.

In one preferred embodiment, the weight ratio of TQ to vitamin D is in the range of 1:0.0001-1:1.

In another preferred embodiment, the weight ratio of TQ to vitamin D is in the range of 1:0.0005-1:0.1.

In yet another preferred embodiment, the weight ratio of TQ to vitamin D is in the range of 1:0.005-1:0.05.

### BRIEF DESCRIPTION OF THE DRAWINGS

FIG. 1 presents, in its lower portion a bar graph showing the effect of various concentrations of TQ on the accumulation of lipids in cultured adipocytes. The upper portion of FIG. 1 presents a series of photomicrographs showing the effect of various TQ concentrations on the accumulation of lipid droplets in the cultured cells.

FIG. 2 presents, in its lower portion, a bar graph showing the effect of different concentrations of vitamin D3 on the average lipid droplet size in cultured adipocytes. The upper part of this figure presents photographs of the stained adipocytes, confirming the numerical results shown in the graph.

FIG. 3 presents, in its lower part, a graph showing the effect of different combinations of TQ and Vitamin D3 on the average droplet size in cultured adipocytes. The photographs of stained cultured cells shown in the upper part of the figure confirm these numerical results.

FIG. 4 presents a series of photomicrographs that demonstrate the increased reduction in lipid droplet size seen when TQ (2 μM) and Vitamin D3 (10 nM) are used in combination, as compared with the same concentrations of TQ and D3 alone.

FIG. 5 is a bar graph present numerical data for the same experiment used to generate the photographic results shown in FIG. 4.

FIG. 6 graphically presents the results of UCP1 expression following treatment with the combination composition, and, separately, with TQ and Vitamin D3 alone.

FIG. 7 graphically presents the results of PGC-1α expression following treatment with the combination composition, and, separately, with TQ and Vitamin D3 alone.

FIG. 8 graphically presents the results of Mfn2 expression following treatment with the combination composition, and, separately, with TQ and Vitamin D3 alone.

FIG. 9 is a bar graph showing the effects of a combination of Vitamin D3 and TQ on the levels of TNF-α produced by cultured adipocytes,

### DETAILED DESCRIPTION OF PREFERRED EMBODIMENTS

As explained hereinabove, the present invention is primarily directed to a composition comprising a combination of thymoquinone (TQ) and vitamin D. This composition may be used to prevent or treat many of the changes associated with metabolic syndrome, such as insulin resistance and increased visceral fat—by means of supporting healthy weight management—as well as preventing inflammation and providing support for the immune system.

In one preferred embodiment of the composition of the invention, said composition comprises TQ and vitamin D at a ratio of 0.5 mg TQ to 250 IU vitamin D. (It is to be noted that, generally, 1 IU of vitamin D has a mass of 0.025 micrograms.)

In one preferred embodiment, the vitamin D used to prepare the composition is vitamin D3. However, in other embodiments, the composition of the present invention may comprise vitamin D2 or a mixture of vitamin D2 and vitamin D3.

In one preferred embodiment of the presently disclosed composition, the TQ is obtained from a plant source. In one particularly preferred embodiment, the TQ is provided in a cold-pressed botanic seed oil. Preferably, this is a cold-pressed seed oil. In a particularly preferred embodiment, the cold-pressed seed oil is Nigella sativa seed oil.

In another preferred embodiment, the TQ used in the composition of the present invention is obtained by means of extraction (e.g., solvent extraction or supercritical CO₂ fluid extraction) from a plant source.

While Nigella sativa is a preferred plant species source of TQ, other plant sources include (but are not limited to): other species of Nigella, and species of the Monarda, Origanum, Thymus, Satureja and Eupatorium genera.

The present invention is further directed to a dosage form comprising a composition as disclosed hereinabove. Preferably, said dosage form comprises at least 15 mg TQ, at least 1,000 IU of vitamin D and one or more excipients or additives. Preferably, the amount of vitamin D in said dosage form is in the range of 1,000 to 5,000 IU. In a preferred embodiment, the dosage form also comprises one or more free fatty acids, wherein the weight ratio of TQ to said free fatty acids is at least 1:1.5, preferably at least 1:1, and more preferably at least 1:0.6. Without wishing to be bound by theory, it is believed that the relatively low concentration of free fatty acids in some of the compositions of the present invention may be responsible, at least in part, for allowing the expression of the synergistic interaction between vitamin D and TQ.

When present in the composition of the present invention, the free fatty acids may be of various different types. In some embodiments, the composition of the present invention may comprise one or more of the following free fatty acids: linoleic acid, oleic acid, palmitic acid and stearic acid.

Preferably, the weight ratio of TQ to vitamin D is such that there is a synergistic interaction between these two agents, measurable in at least one biological model. Appropriate numerical values for this ratio are provided hereinabove.

In one particularly preferred embodiment, the various combinations of TQ and vitamin D of the present invention are administered as a single composition. In other embodiments, the TQ and the vitamin D may be administered separately, in different formulations. Such separate administration of the two components may be either simultaneous or sequential (in either order).

The compositions of the present invention (whether they contain the combination of the two different components, or whether sad components are administered separately) may be administered systemically or topically. In one preferred embodiment, said compositions are administered orally, and for this purpose may be formulated as capsules, caplets, tablets, hard candies, gummies, liquids, taste-masked liquids, syrups and the like.

In another embodiment the compositions may be formulated for topical administration, for example as creams, gels, oils, foams, patches, ointments or lotions for application to the skin or external mucous membranes, or as pessaries for rectal or vaginal administration.

Further details of suitable formulations for use in the present invention are well known to the skilled artisan in this technical field and may be obtained from standard reference works such Remington's Pharmaceutical Sciences, Mack Publishing Co, Easton, Pa, USA (1980).

In one preferred embodiment of the present invention, the dosage form is suitable for oral administration. In one particularly preferred embodiment, the oral dosage form is a soft-gel (e.g., gelatin-based) capsule comprising 500 mg of a cold-pressed Nigella sativa seed oil and 3, 000 IU of vitamin D3. Preferably, said seed oil comprises 3% TQ and 1.8% free fatty acids.

The present invention also provides methods for preventing, inhibiting, reversing or treating various aspects associated with metabolic syndrome. As explained hereinabove, the term “metabolic syndrome” is used to describe a cluster of several different abnormal physical parameters, notably at least three of: abdominal obesity, elevated blood pressure, high blood glucose, high blood triglycerides and low levels of HDL-cholesterol. Metabolic syndrome is often associated with diseases such as type-2 diabetes and coronary heart disease.

Thus, in one aspect, the present invention is directed to a method for providing immune system support in a subject in need thereof, comprising administering to said subject a composition or dosage form of the present invention. It is to be noted that the term “immune system support” is used herein to indicate the modulation of the immune response to an external threat (e.g., a microbial invader) or an internal threat (e.g., neoplastic cellular proliferation) or a perceived internal threat (e.g., auto-immune conditions), thereby assisting the various cellular and chemical mediators of said immune system to act in a balanced way which is optimal for the host subject. The term may also be understood to refer to various possibly subtle modulatory effects of the immune system which are able to compensate for undesirable changes in immune function, such as those which occur as a result of stress, certain long-term drug treatments, as well as age-related immune function decline.

In another aspect, the present invention is directed to a method for preventing or treating an inflammatory disorder in a subject in need thereof, comprising administering to said subject a composition or dosage form of the present invention. The term “inflammatory disorder”, as used herein, is to be understood to refer both to diseases and states which are primarily characterized by an activation of pro-inflammatory mechanisms, and to undesirable inflammatory responses which may accompany other, non-inflammatory pathological processes (such as neoplasia and cell and tissue degeneration).

Non-limiting examples of inflammatory conditions which may be treated, inhibited or prevented with the method of the present invention include: osteoarthritis, rheumatoid arthritis, asthma, rhinitis and other upper respiratory tract inflammatory conditions, adult respiratory distress syndrome (ARDS), cardiovascular inflammation, reperfusion injury, peritonitis, cirrhosis, inflammatory bowel disease, inflammatory skin disorders including psoriasis, bullous diseases, eczema, allergic reactions in the skin, inflammatory conditions of the eyes, retinopathy, inflammatory conditions of infective origin, such as sepsis, trauma and other acute inflammatory conditions, chronic inflammatory conditions, allergies and hypersensitivity reactions of all of the various tissues, organs and organ systems. Many other such inflammatory conditions may also be treated or prevented using the method of the present invention, and therefore within the scope of the present invention as claimed.

In one preferred embodiment of this method of the present invention, the inflammatory disorder to be treated is a disorder mediated, at least in part, by the inflammatory mediator TNF-α.

In another aspect, the present invention is directed to a method for preventing or reversing insulin resistance in a subject in need thereof, comprising administering to said subject a composition or dosage form of the present invention.

In another aspect, the present invention is directed to a method for assisting a subject in obtaining and/or managing a desired body weight, comprising administering to said subject a composition or dosage form of the present invention. The phrase “obtaining and/or managing a desired body weight” includes within its scope the reduction in body weight in overweight or obese individuals (including subjects with metabolic syndrome), as a consequence of a reduction in the amount and percentage of fat stored in adipose tissue.

In some preferred embodiments of the above-disclosed methods, said method provides a daily thymoquinone dosage of about 15-70 mg, a daily vitamin D dosage of about 3,000-8,000 IU and a maximum daily free fatty acid dosage of about 10 mg.

In some preferred embodiments of the above-disclosed methods, said method provides a daily thymoquinone dosage of about 30 mg, a daily vitamin D dosage of about 4,000 IU and a maximum daily free fatty acid dosage of about 20 mg.

While the subject treated in each of the above-disclosed methods may be any mammalian subject, in a preferred embodiment, the subject is a human being.

In another aspect, the present invention is directed to a composition of the present invention comprising TQ and vitamin D, as defined hereinabove, for use as a medicament.

In one embodiment, said medicament is used for supporting the immune system.

In another embodiment, the medicament is used for preventing or treating an inflammatory disorder.

In a further embodiment, the medicament is used for preventing or reversing insulin resistance.

In a still further embodiment, the medicament is used for assisting a subject to obtain, manage or maintain a desired body weight.

All of the various definitions and explanations presented hereinabove in relation to the methods of the present invention also apply to the various uses of the composition as a medicament, as disclosed immediately hereinabove.

### EXAMPLES

### General Methods:

### Materials:

The source of the thymoquinone (TQ) used in the Examples discussed below was a cold-pressed Nigella sativa (Thymoquin®, obtained from TriNutra, Israel) comprising 3% TQ and 1.29% free fatty acids (FFA).

The vitamin D3 preparation used was obtained from DSM (Heerlen, The Netherlands). This preparation (product code 5011574) has a vitamin D3 content of 0.9-1.1 MIU/g.

### Cell Culture and Differentiation:

3T3-L1 murine fibroblasts were obtained from American Type Culture Collection (Rockville, MD, USA). After thawing, cells were resuspended in DMEM, supplemented with 10% heat-inactivated fetal bovine serum (FBS, Invitrogen, Carlsbad, CA, USA) and 1% antibiotic/antimycotic solution (Invitrogen). Culture medium was replaced every 3 days containing 0.25 μM dexamethasone, 10 μg/ml insulin and 0.5 mM 3-isobutyl-1-methylxanthine (Sigma-Aldrich, St. Louis, MO, USA) in DMEM with 10% FBS. Differentiating 3T3-L1 preadipocytes were treated with TQ or Vitamin D3 or combinations thereof for 6 days.

### Oil Red O Staining:

The size of the lipid droplets in differentiated adipocytes (i.e., 3T3-L1 cells prepared as in the previous section) following the various treatments was measured using oil red O staining. Briefly, differentiated adipocytes were washed with phosphate buffered saline (PBS) and fixed in 3.7% formaldehyde for 10 min. Cells were stained with oil red O solution for 30 min at 25° C. Staining was visualized using bright-field microscopy. Lipid droplet size was then measured using Image Pro (advance Imaging Concept, Inc., Princeton, NJ).

### Western Blot Analysis:

The cultured adipocytes were homogenized and lysed in RIPA lysis buffer containing protease and phosphatase inhibitors (Complete TM Mini and PhosSTOPTM, Roche Diagnostics, Indianapolis, IN). Protein samples were separated using 10% sodium dodecyl sulfate-polyacrylamide (SDS) gels and transferred to a nitrocellulose membrane (Bio-Rad, Hercules, CA, USA). After blocking, the membranes were then incubated at 4° C. overnight with the following primary antibodies: anti-UCP-1, anti-PGC1α, anti-Mfn anti-TNF-α, and β-actin. Membrane incubations were carried out using a secondary infrared fluorescent dye conjugated antibody absorbing at both 800 nm and 700 nm. The blots were visualized using an Odyssey Infrared Imaging Scanner (Li-Cor Science Tec) and quantified by densitometric analysis after normalization with β-actin. Results were presented in optical density (O.D.) units as expression of the protein being assayed relative to β-actin expression.

### Statistical Analysis

Statistical significance between experimental groups was determined by ANOVA with Tukey-Kramer post-hoc analysis for comparison between multiple groups (GraphPad Prism). The data are presented as means±SEM and p<0.05 was considered statistically significant.

### Example 1

### Effect of Combinations of TQ and Vitamin D3 on Lipid Droplet Size in Differentiated Cultured Adipocytes

The aim of this study was to investigate the effect of combinations of TQ and vitamin D3 on the accumulation of lipids in cultured adipocytes. For the purpose of this investigation, 3T3-L1 murine fibroblasts were grown and differentiated as described hereinabove. The differentiated cultures were then treated for six days with TO, vitamin D3 or combinations thereof. Following this treatment, the size of the accumulated lipid droplets within the cultured cells were assessed using the oil red O staining method described hereinabove.

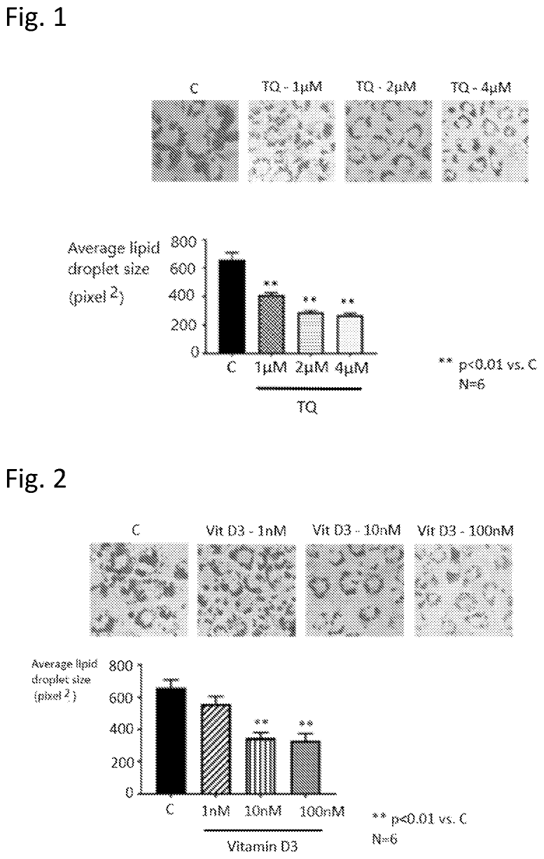

The results for the average lipid droplet size following treatment with 1 μM, 2 μM and 4 μM TQ are presented in the graph shown in FIG. 1. As seen in this graph, TO, at all concentrations tested, caused a statistically significant reduction in lipid droplet size, when compared with the untreated control (C). While the 2 μM concentration of TQ caused a greater degree of size reduction than that caused by the 1 μM dose, increasing the concentration to 4 μM did not cause any further enhancement of this effect.

FIG. 1 also presents photomicrographs of typical areas of the stained adipocytes. The effect of the various TQ concentrations on the accumulation of lipid droplets and the reduction in size of those droplets is clearly seen in these pictures, with the greatest effect being seen with the two higher TQ doses (2 μM and 4 μM).

The results of the average lipid droplet size following treatment with 1 nM, 10 nM and 100 nM Vitamin D3 are shown in the graph presented in the lower portion of FIG. 2. As seen in this graph, Vitamin D3, when used at either 10 nm or 100 nm, caused a statistically significant reduction in lipid droplet size, when compared with the untreated control (C). While the lowest concentration vitamin D3 concentration tested (1 nM) did cause a slight reduction in lipid droplet size, this reduction was not statistically significant.

The photographs of the stained cells in the upper part of FIG. 2 confirm the numerical results shown in the graph.

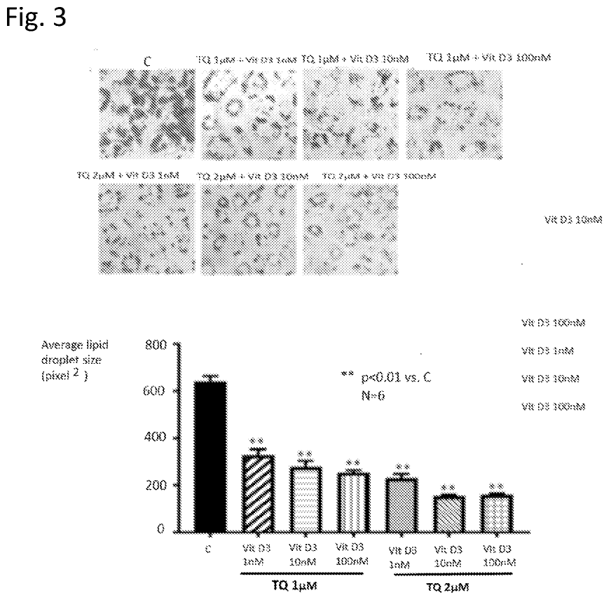

FIG. 3 presents the results obtained with combinations of TQ (1 μM or 2 μM) and Vit D3 (1 nM, 10 nM or 100 nM). The graph in the lower part of the figure shows that at each concentration tested, the combination of the two agents caused a statistically significant reduction in the average lipid droplet size, as compared with the control (C). The overall effect appears generally to be dose-dependent, in relation to both the concentration of TQ and the concentration of vitamin D3. The greatest degree of inhibition of lipid droplet size is found with TQ 2 μM combined with either 10 nm or 100 nM Vitamin D3. These numerical results are confirmed in the stained photomicrographs in the upper part of FIG. 3.

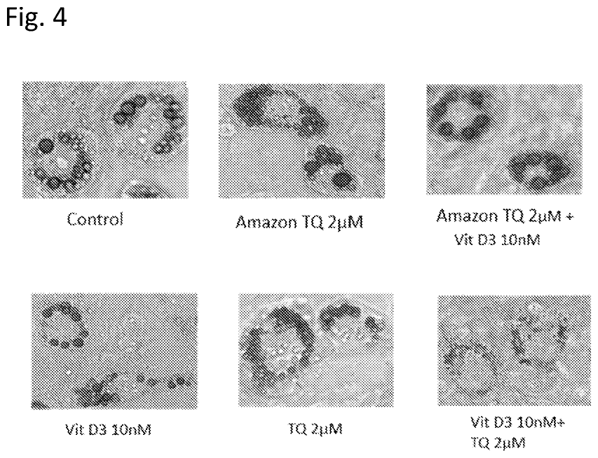

FIG. 4 presents a series of photomicrographs that again show the increased reduction in lipid droplet size seen when TQ (2 μM) and Vitamin D3 (10 nM) are used in combination, as compared with the same concentrations of TQ and D3 alone. Interestingly, when a different commercially-available Nigella sativa oil (“Amazon”) is used as the source of the TO, the same concentration of TQ (2 μM)-either alone or in combination with 10 nM Vitamin D3, there is no decrease in average lipid droplet size. In fact, it would appear from this figure that this TQ source (either alone or in the combination preparation) actually increases the lipid droplet size.

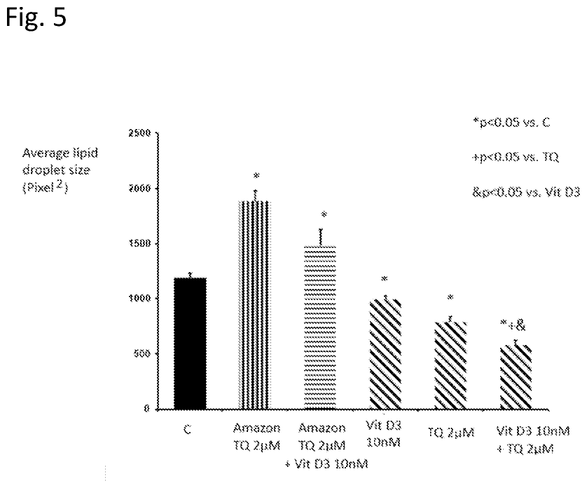

This result is confirmed by the numerical results shown in the graph presented in FIG. 5. In this graph, it may be seen that this alternative (“Amazon”) preparation does indeed cause an increase in droplet size, either alone or in combination with Vitamin D3. It is to be noted that this alternative Nigella sativa oil (obtained from SVA Organics, Delhi, India) contains 1% (w/w) TQ and 8.5% (w/w) free fatty acids. However, the cold-pressed Nigella sativa oil used to generate the other results in this study (and which caused a significant reduction in lipid droplet size, as shown in FIGS. 1 to 5) comprises 3% TQ and 1.29% free fatty acids.

Without wishing to be bound by theory, the present inventors believe that the lower free fatty acid concentration (and thus elevated TQ: free fatty acid ratio) may be, at least in part, an important factor involved in the inhibition of fat droplet accumulation in the adipocytes.

### Example 2

### Effect of Combinations of TQ and Vitamin D3 on the Expression of Genes Involved in the Development of Weight Gain, Obesity, Insulin Resistance and Dysfunctional Glucose Metabolism

The control of the metabolic processes involved in the maintenance of energy levels, blood glucose concentrations and normal body weight is very complex and involves many interacting metabolic pathways, each of which is dependent on the adequate amounts and functions of a very large number of enzymes and co-factors. The effect of various treatments (pharmacological, nutraceutical and others) on different aspects of metabolism can be monitored by measuring the expression of certain key gene products, during or following such treatment. In the present study, the effects of treatment with combinations of TQ and Vitamin D3 on the expression of the following three gene products were measured: UCP1, PGC-1a and Mfn2. The metabolic function and significance of these three proteins may be briefly summarized as follows:

UCP1 (uncoupling protein 1): a mitochondrial protein responsible for thermogenic respiration in brown and beige fat. It is used to generate heat by non-shivering adaptive thermogenesis, and more generally to the regulation of energy balance, by means of uncoupling the respiratory chain, thereby permitting fast substrate oxidation with a relatively low rate of ATP production.

PGC-1a (Peroxisome proliferator-activated receptor gamma coactivator 1-alpha): a coactivator that play a critical role in the maintenance of glucose, lipid and energy homeostasis, and is likely involved in the development of obesity, diabetes, neurodegeneration and cardiomyopathy. PGC-1a is also the primary regulator of liver gluconeogenesis.

Mfn2 (Mitofusin 2): a mitochondrial membrane protein (GTPase) that participates in mitochondrial fusion and contributes to the maintenance and operation of the mitochondrial network. It may play a role in the pathophysiology of obesity. In obesity and type 2 diabetes, MFN2 expression is reduced, this down regulation ultimately leading to insulin resistance.

The aim of this study was to investigate the effect of a combination of vitamin D3 (10 nM) and TQ (1 μM) on the expression of the UCP1, PGC-1α and Mfn2 genes in cultured adipocytes. 3T3-L1 murine fibroblasts were cultured and differentiated as described hereinabove and treated with the test compositions and controls for a period of 6 days. Following SDS-PAGE separation and western blotting, the three gene products were detected using the appropriate antibodies, and the level of each protein measured, all as described above in the general methods section.

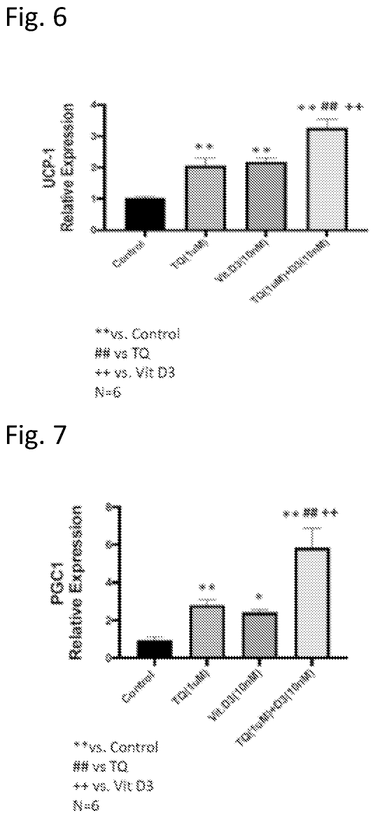

FIG. 6 graphically presents the results of UCP1 expression following treatment with the combination composition, and, separately, with TQ and Vitamin D3 alone. The results in this figure that both TQ and Vit D3, alone, cause statistically significant elevation of UCP1 expression. The combination composition, however, causes a significantly greater elevation of the level of expression than either of the two single-substance treatments.

FIG. 7 graphically presents the results of PGC-1α expression following treatment with the combination composition, and, separately, with TQ and Vitamin D3 alone. The results in this figure that both TQ and Vit D3, alone, cause statistically significant elevation of PGC-1α expression. The combination composition, however, causes a greater-than-additive (i.e., synergistic) elevation of PGC-1α expression than either of the two single-substance treatments. The differences between the combination treatment and both of the single-substance treatments (and likewise, the untreated control cells) were all statistically significant.

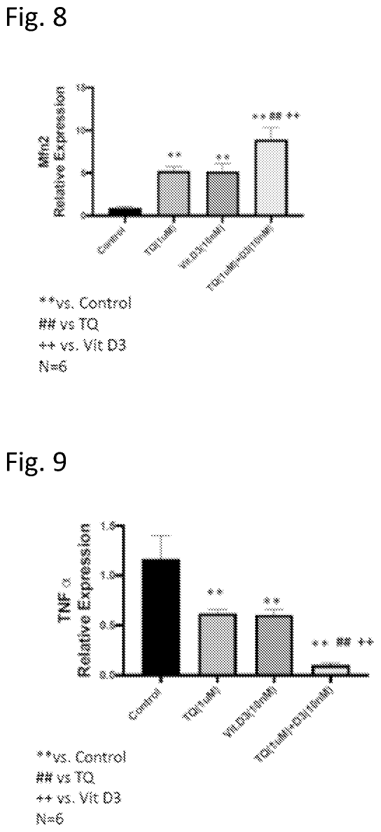

FIG. 8 graphically presents the results of Mfn2 expression following treatment with the combination composition, and, separately, with TQ and Vitamin D3 alone. The results in this figure that both TQ and Vit D3, alone, cause statistically significant elevation of Mfn2 expression, when compared with the untreated control (C). The combination composition, however, causes a significantly greater elevation of the level of expression than either of the two single-substance treatments.

The results of this preliminary study indicate that the composition of the present invention is capable of enhancing the expression of three gene products, each of which are involved in one or more of the regulation of metabolism, glucose levels, insulin sensitivity and maintenance of a healthy body weight.

### Example 3

### Effect of Combinations of TQ and Vitamin D3 on the Expression of TNF-Alpha in Cultured Adipocytes

The aim of this study was to investigate the potential effects of a combination of Vitamin D3 and TQ, according to the present invention, on the levels of TNF-α produced by cultured adipocytes, following treatment with said combination.

TNF-α is an important mediator and modulator of the immune response, including the inflammatory response. For example, TNF-α released by macrophages, e.g., during infection, serves as a messenger to alert and recruit other immune system cells as part of the initial inflammatory response.

The levels of TNF-α produced by cultured, differentiated 3T3-L1 cells were measured using specific anti-TNF-α antibodies in a western blot assay, as described hereinabove. The treatment groups used were as follows: 1 μM TQ alone, 10 nM Vitamin D3 alone and a combination of 1 μM TQ and 10 nM Vitamin D3. The results obtained were compared with those obtained from untreated control cells.

The results of this study are shown in FIG. 9. It may be seen from this figure that both 1 μM TQ and 10 nM Vitamin D3, when tested alone, cause an approximately 50% decrease in TNF-α expression when compared with the untreated control (C). When the combination of these two substances is tested, however, TNF-α expression is inhibited to a far greater extent, such that the expression level approaches zero. The inhibition level caused by the combination treatment is significantly different from the inhibition levels caused by the single-substance treatments, and indicates a synergistic interaction between TQ and vitamin D.

It may be concluded from this study that the combination composition of the present invention possesses significant activity on the immune system and is capable of inhibiting the production of the inflammatory mediator TNF-α to a high degree.

### Example 4

### Soft-Gel Capsule Formulation Containing a Composition of the Present Invention

Each gelatin-based soft-gel capsule contains 500 mg of a cold-pressed Nigella sativa seed oil (ThymoQuin 3%, obtained from N.S. Oils Ltd., Sa′ad, Israel) and 3,000 IU of Vitamin D3. Said N. sativa oil contains 3% w/w thymoquinone and 1.8% w/w free fatty acids.

The formulation is generally administered to subjects at a dose of two capsules per day.

1. A composition comprising thymoquinone (TQ) and vitamin D, wherein the weight ratio of said TQ to said vitamin D is in the range of 1:0.0001-1:1.

2. A composition according to claim 1, wherein the concentration of thymoquinone is at least 2% w/w, and wherein said composition further comprises one or more free fatty acids, the total concentration of said free fatty acids being 3% w/w or less.

3. The composition according to claim 1, wherein the ratio of TQ to vitamin D is such that there is a synergistic interaction between them.

4. The composition according to claim 1, wherein TQ and vitamin D are present at a ratio of 0.5 mg TQ to 250 IU vitamin D.

5. The composition according to claim 1, wherein the vitamin D is vitamin D3 or vitamin D2.

6. The composition according to claim 1, wherein the TQ is obtained from a plant source.

7. The composition according to claim 6, wherein the TQ is provided in a cold-pressed seed oil.

8. The composition according to claim 7, wherein the cold-pressed seed oil is cold-pressed Nigella sativa seed oil.

9. The composition according to claim 1, wherein the TQ is synthetic TQ.

10. A dosage form comprising a composition according to claim 1, wherein said dosage form comprises at least 15 mg TQ, at least 1,000 IU of vitamin D and one or more excipients or additives.

11. The dosage form according to claim 10, wherein said dosage form is suitable for oral administration.

12. The dosage form according to claim 11, wherein said dosage form is a soft-gel capsule comprising 500 mg of a cold-pressed Nigella sativa seed oil and 3,000 IU of Vitamin D3, and wherein said seed oil comprises 3% w/w TQ and 1.8% w/w free fatty acids.

13. A method for supporting the immune system in a subject in need thereof, comprising administering to said subject a composition according to any one of claims 1-9 or a dosage form according to claim 10.

14. A method for preventing or treating an inflammatory disorder in a subject in need thereof, comprising administering to said subject a composition according to claim 1.

15. A method for preventing or reversing insulin resistance in a subject in need thereof, comprising administering to said subject a composition according to claim 1.

16. A method for assisting a subject in managing a desired body weight, comprising administering to said subject a composition according to claim 1.

17. The method according to claim 11, wherein said method provides a daily thymoquinone dosage of about 15-70 mg, a daily vitamin D dosage of about 3,000-8,000 IU and a maximum daily free fatty acid dosage of about 20 mg.

18. The method according to claim 13, wherein said method provides a daily thymoquinone dosage of about 30 mg, a daily vitamin D dosage of about 4,000 IU and a maximum daily free fatty acid dosage of about 20 mg.

19. A composition comprising thymoquinone (TQ) and vitamin D according to claim 1, for use as a medicament.

20. The composition for use according to claim 19, wherein the medicament is used for supporting the immune system.

21. The composition for use according to claim 19, wherein the medicament is used for preventing or treating an inflammatory disorder.

22. The composition for use according to claim 19, wherein the medicament is used for preventing or reversing insulin resistance.

23. The composition for use according to claim 19, wherein the medicament is used for assisting a subject in managing a desired body weight.
